(19) United States
(12) Patent Application Publication
(10) Pub. No.: US 2015/0338400 A1
(43) Pub. Date: Nov. 26, 2015

(54) OPTICAL SENSOR, DETECTION METHOD USING OPTICAL SENSOR, METHOD FOR AFFIXING CAPTURE BODY, AND INSPECTION UNIT

(71) Applicant: Panasonic Intellectual Property Management Co., Ltd., Osaka (JP)

(72) Inventors: Kiyoshi HASHIMOTODANI, Kyoto (JP); Yusuke KITAGAWA, Kyoto (JP)

(21) Appl. No.: 14/410,552

(22) PCT Filed: Jun. 25, 2013
(86) PCT No.: PCT/JP2013/003950

§ 371 Date: Dec. 22, 2014

(30) Foreign Application Priority Data
Jun. 26, 2012 (JP) 2012-142641

(51) Int. Cl. G01N 33/543; G01N 21/45

(52) U.S. Cl. CPC G01N 33/54373; G01N 21/45

(57) ABSTRACT

The optical sensor includes a first metal layer having top and a bottom faces, a second metal layer having top and bottom faces, and a hollow area sandwiched by the first metal layer and the second metal layer. A capturing body for capturing a target substance to be detected can be disposed in the hollow area. Thicknesses of the first metal layer and the second metal layer are both not less than 5 nm and not greater than 50 nm. The hollow area includes a determining part for determining the presence of the target substance contained in a specimen. The second metal layer can transmit an electromagnetic wave from the bottom face to the top face. The first metal layer can transmit the electromagnetic wave from the bottom face to the top face.

### TECHNICAL FIELD

The present invention relates to optical sensors using an optical interference phenomenon typically for detecting viruses, inspection methods using the optical sensor, a method for affixing capture body in the optical sensor, and inspection units.

### BACKGROUND ART

FIG. 12 is a sectional view of optical sensor 100 disclosed in PTL 1 that can be used typically for detecting viruses. Optical sensor 100 includes prism 101, metal layer 102, insulating layer 103, and capturing body 104. Metal layer 102 with flat surface is disposed on a bottom face of prism 101. Insulating layer 103 is disposed on a bottom face of metal layer 102. Insulating layer 103 has a flat surface and a predetermined dielectric constant. Capturing body 104 is, for example, an antibody and is affixed on a bottom face of insulating layer 103.

A surface plasmon-wave, which is a compressional wave of electrons, is present (not illustrated) on a boundary face of metal layer 102 and insulating layer 103. Light source 105 is disposed above prism 101. A p-polarized light enters from light source 105 to prism 101 under a total reflection condition. At this point, an evanescent wave is generated near the boundary face of metal layer 102 and insulating layer 103. Wave detector 106 receives the light totally reflected on metal layer 102, and detects the light intensity.

When the wavenumber matching condition, in which the wavenumber of evanescent wave and the wavenumber of surface plasmon wave match, is satisfied, light energy supplied from light source 105 is used for exciting the surface plasmon wave. This reduces the intensity of reflected light. The wavenumber matching condition depends on an incident angle of light supplied from light source 105. Accordingly, when the incident angle is changed and wave detector 106 detects the intensity of reflected light, the intensity of reflected light reduces at a certain incident angle.

The resonance angle that is an angle at which the intensity of reflected light is minimized depends on a dielectric constant of insulating layer 103. When a bound substance generated by specific binding of analyte, which is a target substance to be detected in a specimen, and capturing body 104 is formed on the bottom face of insulating layer 103, the dielectric constant of insulating layer 103 changes. According to this change of dielectric constant, the resonance angle changes. Accordingly, a binding strength and binding speed in specific binding of analyte and capturing body 104 can be detected by monitoring this change of resonance angle.

Above conventional optical sensor 100 includes light source 105 that can supply a p-polarized light and prism 101 disposed on the top face of metal layer 102. Therefore, optical sensor 100 is large in size and its structure is complicated.

An optical sensor disclosed in PTL 2 is proposed with an aim of achieving a small optical sensor with simple structure.

FIG. 13 is a schematic view of optical sensor 201 disclosed in PTL 2. Optical sensor 201 includes a top face and bottom face configured to supply an electromagnetic wave. The top face has first metal layer 202 formed of metal, such as gold and silver. The bottom face has second metal layer 203 formed of metal, such as gold and silver. The bottom face of first metal layer 202 faces the top face of second metal layer 203. A thickness of first metal layer 202 is not less than 30 nm and not greater than 45 nm. A thickness of second metal layer 203 is 100 nm or more. Hollow area 204 configured to be filled with specimen 208 typically containing solutes 208A, 208B, and 208C is provided between first metal layer 202 and second metal layer 203. Multiple capturing bodies 207 are physically adsorbed at least on the bottom face of first metal layer 202 or the top face of second metal layer 203 in hollow area 204. Light supplied from light source 209, which is one type of electromagnetic source, to first metal layer 202 can generate optical resonance at first boundary face 202B of first metal layer 202 and hollow area 204, and second boundary face 203A of second metal layer 203 and hollow area 204. If solute 208C, which is a target substance (analyte) that specifically binds with capturing body 207, is present in specimen 208, capturing body 207 and analyte specifically bind, and a dielectric constant changes. As a result, resonance absorbing wavelength against the light supplied from light source 209 changes due to a change of the optical resonance condition. A change of resonance absorbing wavelength is visually detected as a color change. Optical sensor 201 does not need a prism. In addition, the light supplied from light source 209 does not need to be in a predetermined polarized state or have coherence. As a result, a small optical sensor with simple structure can be achieved.

### CITATION LIST

### Patent Literature

PTL 1 Japanese Patent Unexamined Publication No. 2005-181296

PTL 2 International Publication No. 2010/122776

### SUMMARY OF THE INVENTION

An optical sensor of the present invention includes a first metal layer having a first top face and a first bottom face, a second metal layer having a second top face and a second bottom face, and a hollow area that is an area sandwiched by the first metal layer and the second metal layer. A capturing body for capturing a target substance to be detected can be disposed in the hollow area. The first bottom face of the first metal layer faces the second top face of the second metal layer. Thicknesses of the first metal layer and the second metal layer are not less than 5 nm and not greater than 50 nm. The hollow area includes a determining part for determining the presence of the target substance contained in a specimen. The second metal layer can transmit an electromagnetic wave from the second bottom face to the second top face. The first metal layer can transmit an electromagnetic wave from the first bottom face to the first top face.

A detection method of the optical sensor of the present invention includes the steps of disposing the capturing body for capturing the target substance in the hollow area of the optical sensor, filling the specimen in the hollow area using capillary action, entering the electromagnetic wave from the second bottom face of the second metal layer, and detecting the electromagnetic wave passing through the first metal layer. The above optical sensor includes the first metal layer having the first top face and the first bottom face, the second metal layer having the second top face and the second bottom face, and the hollow area that is an area sandwiched by the first metal layer and the second metal layer. The capturing body for capturing the target substance can be disposed in the hollow area. In the above optical sensor, the first bottom face of the first metal layer faces the second top face of the second metal layer. Thicknesses of the first metal layer and the second metal layer are not less than 5 nm and not greater than 50 nm. In addition, the hollow area in the optical sensor includes a determining part for determining the presence of the target substance contained in the specimen.

A method for affixing a capturing body of the present invention includes the first step of filling a solute containing the capturing body for capturing the target substance in the hollow area of the optical sensor, and the second step of drying the solute to dispose the capturing body in the hollow area after the first step. The above optical sensor includes the first metal layer having the first top face and the first bottom face, the second metal layer having the second top face and the second bottom face, and the hollow area that is an area sandwiched by the first metal layer and the second metal layer. The capturing body for capturing the target substance can be disposed in the hollow area. The first bottom face of the first metal layer faces the second top face of the second metal layer. Thicknesses of the first metal layer and the second metal layer are not less than 5 nm and not greater than 50 nm. The hollow area includes the determining part for determining the presence of the target substance contained in the specimen.

An inspection unit of the present invention uses an inserted optical sensor. The inspection unit includes an opening, an optical sensor case for receiving the optical sensor inserted from the opening, an electromagnetic source for irradiating the electromagnetic wave to the optical sensor, and an optical path for guiding the light irradiated from the electromagnetic source through the optical sensor at a predetermined angle to outside the inspection unit. The above optical sensor includes the first metal layer having the first top face and the first bottom face, the second metal layer having the second top face and the second bottom face, and the hollow area that is an area sandwiched by the first metal layer and the second metal layer. The capturing body for capturing the target substance can be disposed in the hollow area. The first bottom face of the first metal layer faces the second top face of the second metal layer. Thicknesses of the first metal layer and the second metal layer are not less than 5 nm and not greater than 50 nm. The hollow area includes a determining part for determining the presence of the target substance contained in the specimen. The second metal layer can transmit the electromagnetic wave from the second bottom face to the second top face. The first metal layer can transmit the electromagnetic wave from the first bottom face to the first top face.

With the above structure, the optical sensor of the present invention can position the electromagnetic source in alignment with the detector with the optical sensor in between by the use of a transparent wave instead of reflected wave of the electromagnetic wave supplied from the electromagnetic source. Accordingly, the optical sensor of the present invention enables electromagnetic wave irradiation and observation always at an optimum angle. This prevents any difference in visual color due to a change of the optical resonance condition by angle.

Furthermore, the use of the inspection unit with built-in electromagnetic source, whose propagation path of electromagnetic wave is optimally designed, enables to further optimize the above positions of the electromagnetic source and optical sensor. In addition, the electromagnetic source with spectrum optimized for the optical resonance condition of the optical sensor can always be used. Accordingly, degradation of sensitivity due to types of electromagnetic source can be prevented to further improve the detection sensitivity.

### BRIEF DESCRIPTION OF DRAWINGS

FIG. 1 is a sectional view of an optical sensor in accordance with a first exemplary embodiment.

FIG. 2A is a schematic view illustrating the position of capturing body.

FIG. 2B is a conceptual diagram of specific binding of the capturing body and analyte.

FIG. 3A is a schematic view of aggregation of the capturing body.

FIG. 3B is a schematic view of aggregation of the capturing body in a hollow area.

FIG. 4A is a schematic view of an optical path of transmitted electromagnetic wave in the optical sensor in accordance with the first exemplary embodiment.

FIG. 4B is a schematic view of the optical path of transmitted electromagnetic wave in the optical sensor in accordance with the first exemplary embodiment.

FIG. 5 is a graph illustrating a change of transmission spectrum of the optical sensor due to refractive index in accordance with the first exemplary embodiment.

FIG. 6 is a graph illustrating dependency of refractive index on center wavelength at a peak of transmission spectrum.

FIG. 7 is a graph illustrating a relationship of the center wavelength of peak transmission spectrum and a height of the hollow area.

FIG. 8 is a graph illustrating spectrum of light source against a change of transmission spectrum due to a change of refractive index.

FIG. 9 is schematic view illustrating a structure of an inspection unit in accordance with a fourth exemplary embodiment.

FIG. 10 is a schematic view of an optical system inside the inspection unit in accordance with the fourth exemplary embodiment.

FIG. 11 is a schematic view of the optical system inside the inspection unit in accordance with the fourth exemplary embodiment.

FIG. 12 is a sectional view of a conventional optical sensor.

FIG. 13 is a sectional view of a conventional optical sensor.

FIG. 14 illustrates a relationship of a solute and refractive index.

### DESCRIPTION OF EMBODIMENTS

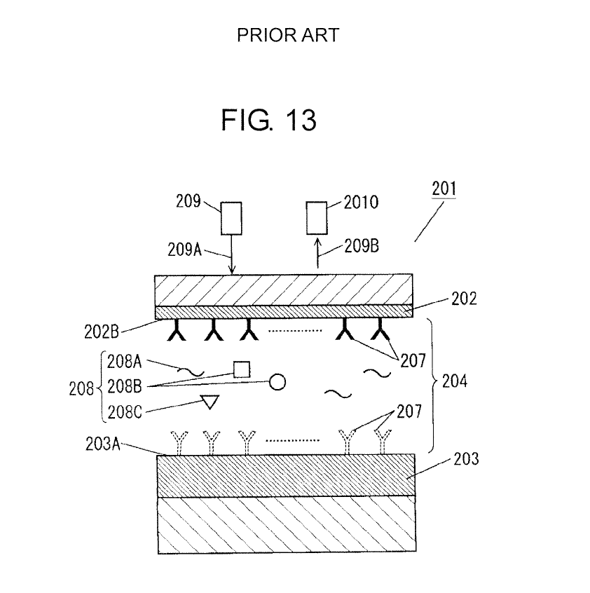

Before describing exemplary embodiments of the present invention, disadvantages in a conventional optical sensor show in FIG. 13 are described.

In optical sensor 201 shown in FIG. 13, detection depends on a color change of reflected light. The color change of reflected light is caused by a change of optical resonance absorption wavelength against light supplied from light source 209, depending on a change of refractive index (can be considered equivalent to a change of electric constant) in hollow area 204 due to the presence of specific binding of capturing body 207 and analyte. However, since a binding amount of analyte in question is an extremely small amount in general biochemical sensors, an amount of change of refractive index corresponding to the binding amount is also not so large in some cases.

Still more, the optical resonance absorption wavelength in reflected light is determined by optical path difference inside the optical sensor. Therefore, the condition changes when an incident angle of light supplied from the light source and an observation angle for observing reflected light change. This may cause a color of reflected light look different, obstructing detection in some cases.

Still more, a change of color due to optical resonance may be difficult to detect depending on spectrum of the light source.

Furthermore, color deficient observers have difficulty in determining any color change in the first place. The presence of detection cannot be detected in some color ranges of reflected light.

Next, exemplary embodiments are described.

### First Exemplary Embodiment

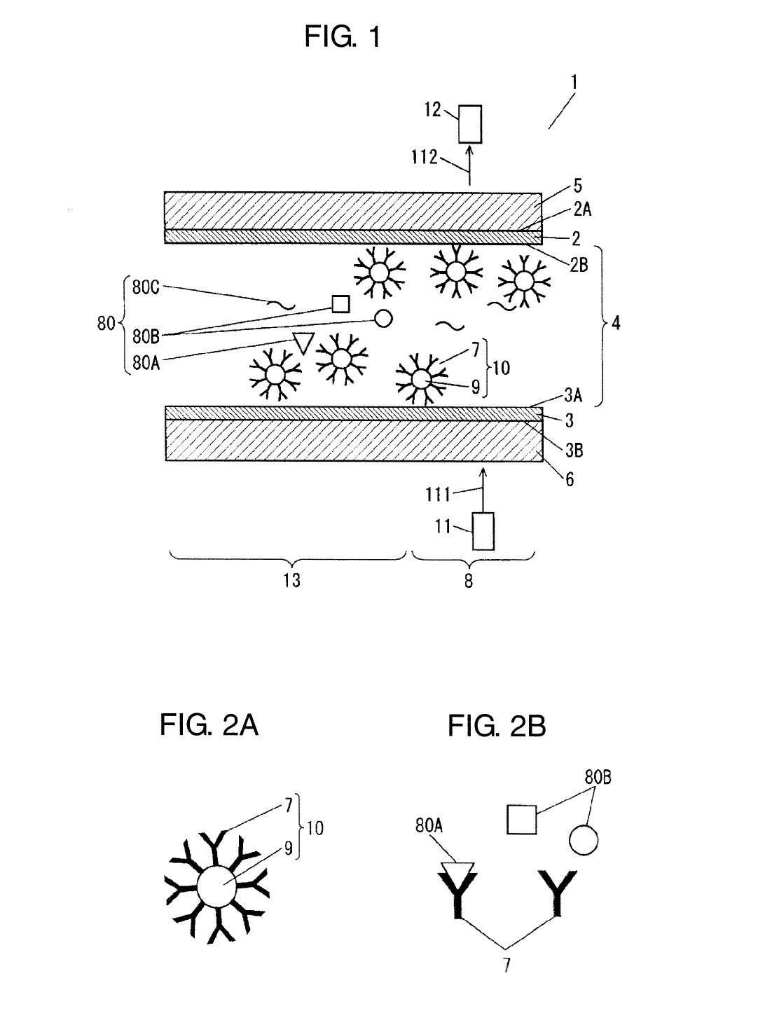

FIG. 1 is a schematic sectional view of optical sensor 1 in the first exemplary embodiment of the present invention. Optical sensor 1 includes metal layer 2 (first metal layer), metal layer 3 (second metal layer), and hollow area 4. Metal layer 2 has top face 2A and bottom face 2B. Metal layer 3 has top face 3A and bottom face 3B. Top face 3A of metal layer 3 faces bottom face 2B of metal layer 2. An area sandwiched by metal layer 2 and metal layer 3 is hollow area 4. Thicknesses of metal layer 2 and metal layer 3 are not less than 5 nm and not greater than 50 nm. Capturing body 7 that specifically binds with analyte (target substance to be detected) 80A is disposed in hollow area 4.

Determining part 8 for determining a presence of analyte 80A specifically bound with capturing body 7 is provided in hollow area 4.

Capturing body 7 captures predetermined analyte 80A. In other words, capturing body 7 is a substance that specifically binds with analyte 80A. For example, capturing body 7 is antibody, acceptor protein, aptamer, porphyrin, or polymer generated by the molecular imprinting technology.

Analyte 80A is contained in specimen 80 together with other solute 80B, such as protein, and solvent 80c that is mainly water. It is a substance that specifically binds with capturing body 7.

When specimen 80 contains analyte 80A, analyte 80A specifically binds with capturing body 7 and forms an aggregate in determining part 8.

Incident electromagnetic wave 111 supplied from electromagnetic source 11, such as light source, enters from bottom face 3B of metal layer 3 below determining part 8. Then, incident electromagnetic wave 111 passes through metal layer 2 (first metal layer) above determining part 8. Transmitted electromagnetic wave 112 is then detected by detector 12.

As described later, when specimen 80 contains analyte 80A, refractive index in the hollow area changes, but when specimen 80 does not contain analyte 80A, the refractive index in the hollow area does not change. Therefore, the presence of analyte 80A can be determined by detecting a change of refractive index in the hollow area by detector 12.

Metal layer 2 and metal layer 3 are configured typically with gold, silver, or aluminum. When metal layer 2 and metal layer 3 are configured with gold, reflectivity of wavelengths shorter than around 550 nm reduces. This low peak does not positively contribute to detection. Therefore, lower intensity enables to improve color purity at a peak of 500 nm or above that contributes to detection.

Metal layer 3 is not less than 5 nm and not greater than 50 nm in thickness, so as to transmit incident electromagnetic wave 111. Metal layer 3 of this thickness is difficult to retain its shape at its own. Therefore, attaching part 6 is affixed onto metal layer 3 to retain the shape of metal layer 3. Attaching part 6 needs to efficiently supply incident electromagnetic wave 111 to metal layer 3, and thus it is formed of a material that does not easily attenuate incident electromagnetic wave 111. Since incident electromagnetic wave 111 is a visible light (electromagnetic wave with wavelength in a range roughly between 350 nm and 800 nm), attaching part 6 is formed of a transparent material that efficiently transmits visible light, such as a glass and transparent plastic. The thickness of attaching part 6 is preferably as thin as possible within an allowable mechanical strength.

Metal layer 2 has the thickness of not less than 5 nm and not greater than 50 nm, same as metal layer 3. Metal layer 2 is affixed onto attaching part 5 to retain the shape of metal layer 2. Same as attaching part 6, attaching part 5 is formed of a transparent material that efficiently transmits visible light, such as transparent glass and transparent plastic. The thickness of attaching part 5 is also preferably as thin as possible within an allowable mechanical strength.

Incident electromagnetic wave 111 with wavelength in a visible region enters from bottom face 3B of metal layer 3. Since metal layer 3 is sufficiently thin, incident electromagnetic wave 111 passes through metal layer 3, propagates in hollow area 4, and reaches metal layer 2.

Metal layer 2 also preferably has a film thickness of not greater than 50 nm, same as metal layer 3. If metal layer 2 has the film thickness greater than 50 nm, incident electromagnetic wave 111 cannot pass through metal layer 2. Then, no sufficient amount of transmitted electromagnetic wave 112 reaches detector 12. This degrades the sensitivity of optical sensor 1.

Both metal layer 2 and metal layer 3 preferably have the film thickness not greater than 30 nm. By setting the film thickness of not greater than 30 nm to both metal layer 2 and metal layer 3, an appropriate intensity that does not cause too-strong interference in hollow are 4 can be achieved. As a result, the width and intensity of spectrum that appears in transmitted electromagnetic wave 112 become sufficient for detecting a change of refractive index at high sensitivity.

In addition, both metal layer 2 and metal layer 3 preferably have the thickness of not less than 5 nm. A film thickness thinner than this will sharply reduce reflectivity. This decreases interference in hollow area 4 and increases a percentage of incident electromagnetic wave 111 directly passing through without causing interference. The sensitivity of optical sensor 1 all the same reduces.

Optical sensor 1 may have a pillar or wall (not illustrated) for retaining metal layer 2 and metal layer 3 in order to maintain a constant distance between metal layer 2 and metal layer 3. This structure can further secure hollow area 4 in optical sensor 1.

Multiple capturing bodies 7 are affixed onto a surface of particle 9 formed typically of metal or resin. Composite body 10 is formed of capturing bodies 7 and particles 9.

FIG. 2A is a schematic view of composite body 10 in which capturing bodies 7 are affixed on the surface of particle 9. As shown in FIG. 2A, capturing bodies 7 are chemically adsorbed on the surface of particle 9 to form composite body 10. Particle 9 is, for example, polystyrene latex resin with 100-nm diameter. Although the method for affixing is not limited, capturing bodies 7 can be affixed, for example, by chemical adsorption. Capturing bodies 7 may be affixed onto particle 9 by silane coupling treatment or via self-assembled monomolecular membrane.

Multiple composite bodies 10 are disposed in hollow area 4.

For example, affixing part 13 for affixing composite bodies 10 is provided between an area where specimen 80 is injected into determining part 8. Composite body 10 is disposed on affixing part 13 by physical adsorption.

In other words, hollow area 4 preferably includes affixing part 13 and determining part.

The surface of affixing part 13 where composite bodies 10 are affixed is configured with, for example, high polymer, polymer, metal ceramics, glass, or silicon. However, the material is not particularly limited. With respect to ease of processes, affixing part 13 is preferably formed integrally with metal layer 3 formed in determining part 8.

Or, capturing body 7 is physically adsorbed, for example, on at least bottom face 2B of metal layer 2 or top face 3A of metal layer 3 in determining part 8. In this case, multiple capturing bodies 7 may be provided without being aligned, at least below bottom face 2B of metal layer 2 or above top face 3A of metal layer 3.

Since composite bodies 10 are physically adsorbed on the surface of metal layer 2 or metal layer 3, they are easily released from the surface and dispersed again in specimen 80 when specimen 80 is filled from outside.

FIG. 2B schematically illustrates how capturing body 7 and analyte 80A in specimen 80 specifically bind. Capturing body 7 has specificity only to analyte 80A, and thus it binds with analyte 80A in specimen 80 but not with other solute 80B. Using this effect, a predetermined target substance to be measured, such as viral antigen and disease marker protein, can be selectively captured.

By retaining capturing bodies 7 in the state affixed onto particle 9, a structure that makes capturing bodies 7 easier to contact analyte 80A can be achieved when composite bodies 10 are dispersed in specimen 80 again. Therefore, capturing body 7 and analyte 80A efficiently achieves specific binding.

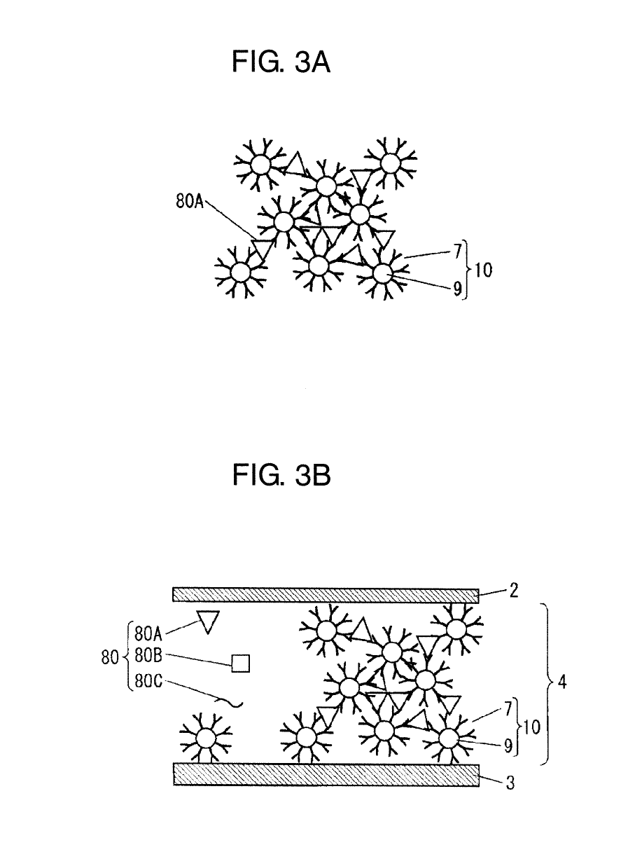

FIG. 3A is a schematic view of an example of aggregate formed by specific binding of capturing bodies 7 and analytes 80A.

Normally, analyte 80A has multiple binding sites to specifically bind with capturing body 7. Therefore, as shown in FIG. 3A, capturing body 7 on particle 9 can bind with another capturing body 7 on particle 9 via analyte 80A. In other words, composite bodies 10 mutually bind linked by analyte 80A to form a collective body (aggregate) of composite bodies 10.

Here, if polystyrene latex resin is used as particle 9, refractive index of polystyrene latex is 1.59.

On the other hand, when solvent 80C in specimen 80 is water, its refractive index is 1.3334.

When specimen 80 contains analyte 80A that specifically binds with capturing body 7, an aggregate of composite bodies 10 is formed as described above. As shown in FIG. 3B, refractive index in determining part 8 increases when the aggregates fill at least a part of determining part 8. As a result, the optical resonance condition in determining part 8 changes. On the other hand, when specimen 80 does not contain analyte 80A, the collective body (aggregate) of composite bodies 10 is not formed. Therefore, refractive index of hollow area 4 is equivalent to solvent 80C, i.e., water. Strictly speaking, the refractive index slightly differs from the case of only water, depending on the concentration of composite bodies 10, because dispersed composite bodies 10 exist. However, unless it is a high concentration close to the emulsion state, this difference can be practically ignored. Accordingly, if a change of the optical resonance condition can be noticed in some way, the presence of analyte 80A in specimen 80 can be detected.

As a material of particle 9, general polystyrene latex resin is used. However, other materials with large difference in refractive index with water can be used. In addition, particle 9 may also be configured with inorganic substances, such as metal oxide, metal, magnetic material, and dielectric material. It may also be configured with an organic substance, such as dendrimer. For example, if microparticles of titanic oxide, which is metal oxide, are used, refractive index further becomes as large as 2.5 or above. A shift amount of resonance wavelength becomes large, and thus further higher sensitivity can be expected.

When particle 9 is configured with a magnetic material, capturing body 7 can be agitated by applying a magnetic field from outside optical sensor 1 after filling specimen 80 in hollow area 4. Capturing body 7 and analyte 80A thus can efficiently achieve specific binding.

If particle 9 is dendrimer, variations in shapes of particles 9 can be reduced because dendrimer can make the shape uniform. Accordingly, variations in performance of optical sensor 1 can be reduced.

Particle 9 in the exemplary embodiment is a globular bead, but other solid shapes are also applicable. For example, if particle 9 is cubic, a filling rate of aggregated particles 9 (composite body 10) in hollow area 4 increases, compared to that of globular particles 9. A calculated filling rate will be 100%, if sizes of capturing body 7 and analyte 80A are ignored. In case of globular particle, the filling rate is 74% in the closest packing.

In the exemplary embodiment, a diameter of particle 9 is 100 nm. However, the exemplary embodiment is not limited to this size. In general, particle 9 can be inserted into hollow area 4 if the size of particle 9 is about a half of the height of hollow area 4. In addition, if the diameter of particle 9 is smaller than 50 nm, the Mie scattering effect reduces and particle 9 can be considered as almost transparent against visible light. Therefore, even a basically opaque material does not hinder propagation of visible light in hollow area 4, and thus it can be used.

Optical sensor 1 in the exemplary embodiment has a characteristic that the optical resonance wavelength changes by a change of refractive index (refractive index n has a relationship of n=∈^{1/2} with respect to dielectric constant ∈, and thus the change of refractive index is equivalent to the change of dielectric constant) in hollow area 4 (determining part 8) between metal layer 2 and metal layer 3. Therefore, capturing bodies 7 do not need to be firmly affixed on metal layer 2 or metal layer 3 typically by chemical adsorption.

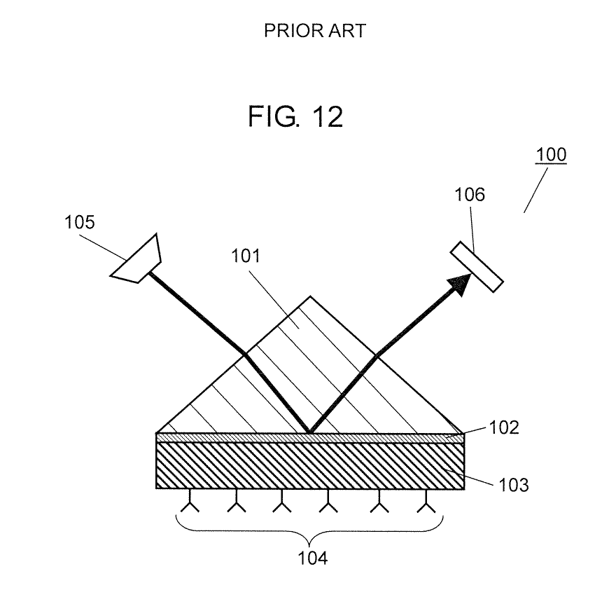

On the other hand, in conventional optical sensor 100 shown in FIG. 12, for example, capturing bodies 104 need to be affixed on the bottom face of insulating layer 103 typically by chemical adsorption in order to secure sensitivity.

Therefore, optical sensor 1 in the exemplary embodiment can simplify the positioning process of capturing bodies 7, such as SAM film formation process. This improves manufacturing efficiency.

In the above description, composite body 10 is disposed on determining part 8 or affixing part 13 of hollow area 4. However, if composite body 10 is disposed in determining part 8, affixing part 13 is not needed.

Next, the operation of optical sensor 1 is described. In the exemplary embodiment, incident electromagnetic wave 111 is a visible light, and electromagnetic source 11 is a visible light source. The light source can be typically an incandescent bulb, halogen lamp, a range of discharge lamps, or sunlight. Electromagnetic source 11 is not equipped with a device to align polarized waves of light, such as a polarizer. Unlike conventional optical sensor 100 shown in FIG. 12, optical sensor 1 in the exemplary embodiment can optically resonate s-polarized light and non-polarized light in addition to p-polarized light.

The wavelength of incident electromagnetic wave 111 that generates optical resonance is controllable primarily by adjusting at least a distance between metal layer 2 and metal layer 3 or effective refractive index in hollow area 4 (determining part 8) between metal layer 2 and metal layer 3.

Capturing bodies 7 on the surface of particle 9 are considered not practically contributing to the refractive index in determining part 9. Here, the effective refractive index in determining part 8 is determined by distribution of refractive index of specimen 80 and refractive index of particles 9 of composite bodies 10 in determining part 8. Accordingly, the effective refractive index in determining part 8 is an average refractive index in a spatial scale equivalent to or higher than wavelengths of incident electromagnetic wave 111 and transmitted electromagnetic wave 112 on a propagation path of incident electromagnetic wave 111 and transmitted electromagnetic wave 112 in determining part 8 of hollow area.

Detector 12 for detecting transmitted electromagnetic wave 112, which is a visible light, is disposed above top face 2A of metal layer 2. Detector 12 receives transmitted electromagnetic wave 112 that has passed through optical sensor 1 after optical sensor 1 receives incident electromagnetic wave 111 given from electromagnetic source 11, which is the light source. In the exemplary embodiment, detector 12 is visual inspection by eyes. However, it may also be a light detector with spectroscopic function.

With this structure, incident electromagnetic wave 111, which is a light supplied from electromagnetic source 11, generates optical resonance (interference) in hollow area 4. Its resonance wavelength is determined by the height of hollow area 4 (determining part 8) and the effective refractive index in hollow area 4 (determining part 8).

Specimen 80 is filled in hollow area 4 of optical sensor 1, and particles 9 on which capturing bodies 7 are affixed, i.e., composite bodies 10, are dispersed again in specimen 80. When composite bodies 10 change to mutually aggregated state via analytes 80A, the resonance wavelength that is optical resonance of optical sensor 1 changes. The formation of aggregates of composite bodies 10 changes the effective refractive index between metal layer 2 and metal layer 3 (determining part 8 of hollow area 4), and thus the resonance wavelength of optical resonance in optical sensor 1 changes.

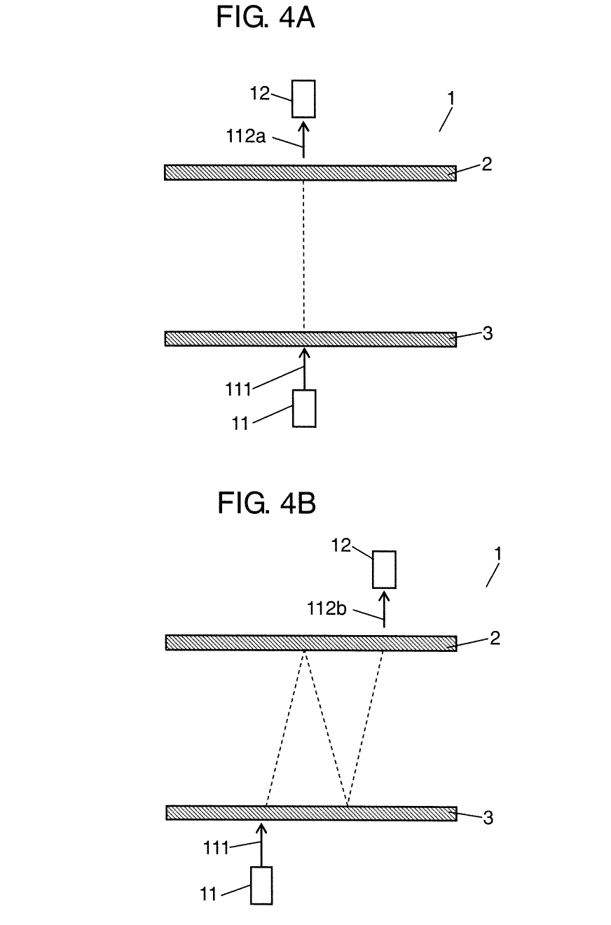

FIGS. 4A and 4B are schematic diagrams illustrating a path of electromagnetic wave from electromagnetic source 11 to detector 12. As shown in FIGS. 4A and 4B, there are at least two types of transmitted electromagnetic wave 112. As shown in FIG. 4A, one is transmitted electromagnetic wave 112a directly passing through metal layer 3 and metal layer 2 and reaching detector 12. As shown in FIG. 4B, the other is transmitted electromagnetic wave 112b that is reflected on bottom face 2B of metal layer 2 after passing through metal layer 3, reflected on top face 3A of metal layer 3 again, and then passing through metal layer 2 to reach detector 12.

Incident electromagnetic wave 111 reflected on metal layer 2 is reflected on metal layer 3 again, and causes interference with subsequent incident electromagnetic wave 111 passing through metal layer 3.

In other words, transmitted electromagnetic wave 112a and transmitted electromagnetic wave 112b interfere. Optical path difference 6 between transmitted electromagnetic wave 112a and transmitted electromagnetic wave 112b can be expressed by the following formula.

δ=2×n×d×cos θ Formula (1)

Whereas, d is a distance between bottom face 2B of metal layer 2 and top face 3A of metal layer 3, which is the height of determining part 8 of hollow area 4, n is the effective refractive index in determining part 8 of hollow area 4, and θ is an incident angle of incident electromagnetic wave 111 measured in the vertical direction relative to metal layer 2. When the optical path difference is the whole-number multiple of the wavelength of incident electromagnetic wave 111, which means m is a whole number larger than 1, phases of transmitted electromagnetic wave 112a and transmitted electromagnetic wave 112b match at wavelength λ satisfying the following formula.

2×n×d×cos θ=mλ Formula (2)

Therefore, the intensity observed by detector 12 becomes the maximum. On the other hand, the intensity does not increase in other wavelengths, and is attenuated in repetitive reflection between metal layer 2 and metal layer 3. When metal layer 2 and metal layer 3 have sufficient thicknesses, detector 12 detects only the wavelength amplified by practically satisfying Formula (2) as transmitted electromagnetic wave 112. In fact, a peak transmission spectrum centering on the wavelength satisfying the condition of Formula (2) is observed in detector 12. This is essentially the multiple reflection interference identical to the Fabry-Perot interference.

The above exemplary embodiment refers to an example of interference between transmitted electromagnetic wave 112a passing through metal layer 3 and metal layer 2 and reaching detector 12 after entering optical sensor 1 and transmitted electromagnetic wave 112b being reflected on metal layer 2 and metal layer 3 twice and reaching detector 12. However, in general, the same discussion applies to a pair of transmitted electromagnetic wave 112a and transmitted electromagnetic wave 112b reaching detector 12 after any different even numbers of reflection.

It is apparent from Formula (2) that the wavelength of incident electromagnetic wave 111 that generates optical resonance in determining part 8 depends on the refractive index in determining part 8. Accordingly, the interference condition, which is a wavelength condition that transmitted electromagnetic wave 112 shows the maximum intensity in the peak transmission spectrum, changes according to a change of effective refractive index in determining part 8. Therefore, the change of effective refractive index in determining part 8 of optical sensor 1 can be detected as a change of wavelength where the transmission spectrum of transmitted electromagnetic wave 112 reinforced by optical resonance reaches its peak, or as a change of color. The change of effective refractive index in determining part 8 is, as described above, caused by aggregation of composite bodies 10. Accordingly, the presence of analyte 80A in specimen 80 can be detected by the change of wavelength of transmitted electromagnetic wave 112.

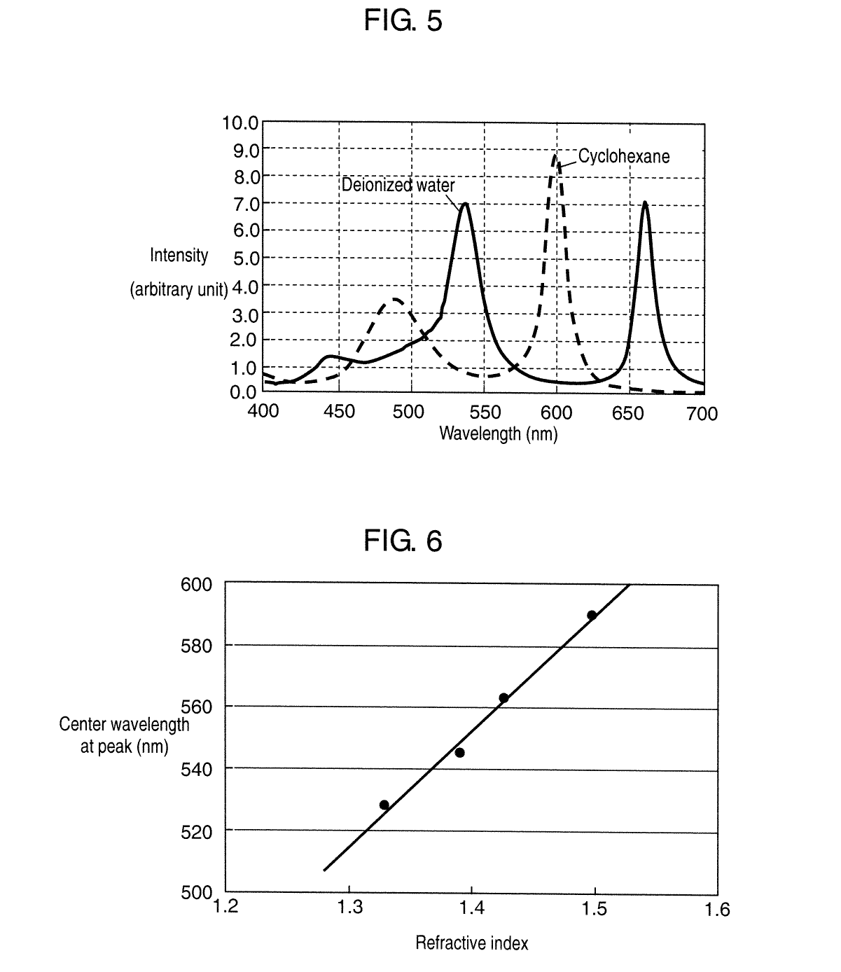

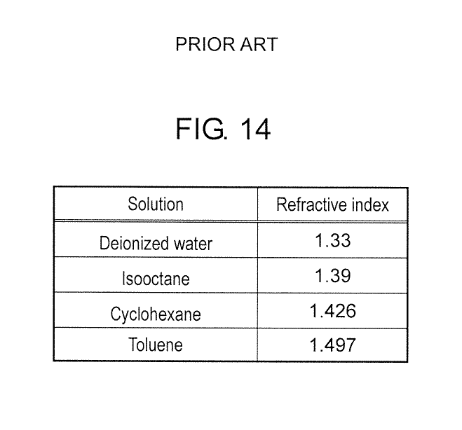

A trial model of optical sensor 1 is manufactured, and FIG. 5 shows spectrums of transmitted electromagnetic wave 112 when a halogen lamp is used as electromagnetic source 11. The height of determining part 8 (distance between bottom face 2B of metal layer 2 and top face 3A of metal layer 3) is 850 nm, and thicknesses of metal layer 2 and metal layer 3 are 20 nm. Two spectrums shown by a solid line and dotted line are results of changing the effective refractive index in hollow area 4 by filling a known standard solution with refractive indexes shown in a table in FIG. 14. The solid line in FIG. 5 is data taken when deionized water (refractive index: 1.33) is used, and the dotted line in FIG. 5 is data taken when cyclohexane (refractive index: 1.426) is used.

In the spectrum shown by the solid line in FIG. 5 (effective refractive index in determining part 8 is 1.33), a peak of transmitted electromagnetic wave 112 appears in multiple wavelengths satisfying Formula (2). These peaks seem to almost correspond to m=3, 4, and 5 from longer wavelength, respectively. When the effective refractive index in determining part 8 increases to 1.426, two peaks out of three to the shorter wavelength side in the spectrum shown by the solid line are shifted for 50 to 60 nm, in line with a change of refractive index, to the longer wavelength side, respectively, as shown by the dotted line. It is apparent that a change of effective refractive index in determining part 8 is successfully detected by a change of peak wavelength.

FIG. 6 is a graph illustrating dependency of the center wavelength at the peak of transmission spectrum on the refractive index. FIG. 6 shows a relationship of the center wavelength at the peak of transmitted electromagnetic wave 112 and refractive index in determining part when a standard refractive index solution of known refractive index is filled in the hollow area. Here, four types of standard refractive index solution shown in the table in FIG. 14 are used.

As shown in FIG. 6, the change of center wavelength is approximately linear to the refractive index.

Although continuous spectrum over the entire visible region enters from electromagnetic source 11, which is the halogen lamp, as incident electromagnetic wave 111 in optical sensor 1, transmitted electromagnetic wave 112 detected by detector 12 is multiple peak spectrums of pseudo-monocolor with a certain bandwidth.

If transmitted electromagnetic wave 112 shows the peak spectrum, it means that a characteristic color with high color purity is present when detector 12 is observation by eyes. In FIG. 5, multiple peaks are noticed in a visible region, but actual color of transmitted electromagnetic wave 112 when seen by eyes is green for the spectrum in the solid line and orange for the spectrum in the dotted line, based on the sensitivity distribution of human eyes (called a spectral sensitivity curve with a peak at 555 nm).

Conventional optical sensor 201 disclosed in PTL 2 determines the presence of specific binding of capturing body 207 and analyte 208C based on detection of a change of resonance absorption wavelength seen in the reflected light in line with a change of refractive index in hollow area 204. Therefore, to increase the sensitivity, a faint change of resonance absorption wavelength needs to be distinguished. This requires a sharp resonance absorption peak, and thus the film thickness of metal layer 202 needs to be as thick as possible within a range that it can retain transmittance of electromagnetic wave 209A. In this case, one needs to read a color tone change (e.g., a change from reddish gold to greenish gold) caused by losing a wavelength band of narrow peak resonance absorption from a gold reflected color spectrum (so-called gold).

Compared to the conventional system, the use of pseudo monochromatic spectrum as detection standard, as in the structure of this exemplary embodiment, achieves reflected color close to monocolor in each refractive index. Accordingly, a color change is easily distinguishable.

For detecting the color change as described above, it is important that the color does not change due to other reasons except for a change of effective refractive index in determining part 8. It is apparent from Formula (2) that the wavelength of transmitted electromagnetic wave 112, whose intensity increases by optical resonance in optical sensor 1, depends on incident angle θ in addition to effective refractive index n in determining part 8. In other words, if the incident angle of incident electromagnetic wave 111 or observation angle of transmitted electromagnetic wave 112 seen from detector 12 changes, the wavelength detected as a peak at detector 12 changes, and a color will look different.

In conventional optical sensor 201 disclosed in PTL 2, the reflected wave of electromagnetic source 209 entering from above optical sensor 201 is also observed based on information of optical sensor 201. In this conventional structure, it is difficult to precisely control angles of incident electromagnetic wave 209A and reflected electromagnetic wave 209B. Since it is impossible to dispose detector 210 and electromagnetic source 209 on the same line, a complicated design will be needed in order to correct incident angle θ.

In contrast, the exemplary embodiment shown in FIG. 4A is configured such that the electromagnetic wave passes through optical sensor 1. This facilitates alignment of electromagnetic source 11 and detector 12 on strictly the same line. Accordingly, an unintended change of wavelength of transmitted electromagnetic wave 112, i.e., change of detected color, at detector 12 can be suppressed.

In the exemplary embodiment, thicknesses of metal layer 2 and metal layer 3 are not less than 5 nm and not greater than 50 nm A qualitative influence of this thickness is as follows.

First, if metal layer 2 and metal layer 3 are thick, reflectivity on metal layer 2 and metal layer 3 increases. Therefore, a wavelength component reflected on metal layer 2 and metal layer 3 that contributes to optical interference relatively increases. On the other hand, transmittance of wavelength components not contributing to interference (i.e., wavelength components not satisfying Formula (2)) reduces when metal layer 2 and metal layer 3 become thick. In other words, the intensity of wavelength components reaching detector 12 but not contributing to interference reduces. Transmittance of wavelength components satisfying Formula (2) also naturally reduces when the layers become thick. Therefore, although an absolute intensity of transmitted electromagnetic wave 112 reaching detector 12 reduces, a baseline of spectrum is relatively lowered because components not satisfying Formula (2) are not transmitted. Therefore, wavelength components satisfying Formula (2) increase. As a result, transmitted electromagnetic wave 112 achieves a sharp and high peak.

Contrarily, when metal layer 2 and metal layer 3 are thin, an absolute intensity of transmitted electromagnetic wave 112 reaching detector 12 increases because the transmittance of optical sensor 1 increases. However, due to an opposite reason to the above, transmittance of wavelength components not satisfying Formula (2) increases as an increase of components satisfying Formula (2) due to interference is relatively reduced. Accordingly, an overall baseline of spectrum becomes high and thus the peak becomes low and broad.

When external light (e.g., sunlight) or a light source with relatively low output, such as an incandescent bulb, is used as electromagnetic source 11, the intensity of transmitted electromagnetic wave 112 decreases if metal layer 2 and metal layer 3 are too thick. This results in difficulty in detecting a change. Accordingly, in this case, the film thickness is set thinner than aforementioned thickness, preferably not less than 5 nm and not greater than 30 nm.

Contrarily, when a light source with intensity stronger than external light (e.g., laser diode) is used as electromagnetic source 11, the film thicknesses of metal layer 2 and metal layer 3 are thickened so that interference is intensified to narrow the peak width of transmission spectrum. This increases the sensitivity.

In the exemplary embodiment, metal layer 2 and metal layer 3 are formed of gold deposited films. In this case, reflectivity of short wavelength from around 550 nm reduces. In FIG. 5, the peak intensity at the shortest wavelength side is low and broad due to this influence, compared to other peaks. This low peak does not actively contribute to detection. Therefore, reduction of intensity at this peak is preferable for improving color purity at peaks over 500 nm that contribute to detection. However, a thick metal film other than gold, such as silver and aluminum, is also applicable. If they are used, cost may be reduced, compared to gold, and they may also be advantageous for using a peak of short wavelength for detection.

A filter may be provided for removing two peaks other than a peak around 550 nm, which mainly contributes to a color change, in multiple peaks in the spectrum indicated by the solid line in FIG. 5. This can further increase the color purity of transmitted electromagnetic wave 112 detected by detector 12, and thus improves the sensitivity.

Next, an example of detection method in the optical sensor in the exemplary embodiment is described.

In the exemplary embodiment, an optical sensor described next is prepared as the first step. The optical sensor prepared includes metal layer 2 whose thickness is not less than 5 nm and not greater than 50 nm, metal layer 3 whose thickness is not less than 5 nm and not greater than 50 nm, and hollow area 4 sandwiched by metal layer 2 and metal layer 3. Top face 3A of metal layer 3 faces bottom face 2B of metal layer 2. Incident electromagnetic wave 111 is supplied from bottom face 3B of metal layer 3. Hollow area 4 includes determining part 8 that can determine the presence of analyte 80A contained in specimen 80 and binding with capturing body 7. Optical sensor 1 as configured above is prepared.

Next is described the second step. In the second step, a solute containing particles 9 onto which capturing bodies 7 are affixed using capillary action, i.e., composite bodies 10, is filled in hollow area 4.

Finally, in the third step, the capturing body is affixed.

How to affix the capturing body is detailed below.

After the second step, the solute containing composite bodies 10 is dried by means such as vacuum-freeze drying. As a result, in the third step, composite bodies 10 are provided in hollow area 4 in a dispersed state.

Composite bodies 10 are provided in determining part 9 or affixing part 13 of hollow area 4.

In optical sensor 1, capturing bodies 7 are not need to be affixed by chemical adsorption in hollow area 4. Therefore, capturing bodies 7 can be disposed in hollow area 4 using aforementioned simple method after combining metal layer 2 and metal layer 3 typically via a pillar for securing and retaining hollow area 4. This enables to efficiently operate optical sensor 1.

Still more, hollow area 4 may be provided in roughly the entire area (including an area where capturing bodies 7 are not provided) between metal layer 2 and metal layer 3. In addition, hollow area 4 may be provided in an area of metal layer 2 and metal layer 3 other than pillar or wall for supporting metal layer 2 and metal layer 3 (including an area where capturing bodies 7 are not provided).

Furthermore, an anti-corrosion coating layer may be applied to bottom face 2B of metal layer 2 and top face 3A of metal layer 3. In this case, hollow area 4 may be provided in an area between metal layer 2 and metal layer 3 other than an area of the anti-corrosion coating layer (excluding an area of capturing bodies 7 disposed on a surface that does not contact metal layer 2 or metal layer 3 with anticorrosion coating agent). Hollow area 4 is an area where specimen 80 can be filled, and this hollow area 4 is secured in a part of area between metal layer 2 and metal layer 3.

A distance between metal layer 2 and metal layer 3, which is a height of determining part 8 of hollow area 4, is preferably within a range of not less than 400 nm and not greater than 1600 nm.

By setting the height of determining part 8 in this range, analyte 80A specifically binds with capturing body 7, and a peak of transmission spectrum, before and after the refractive index of determining part 8 changes, changes across a yellow band of not less than 570 nm and not greater than 590 nm.

In this way, a reflected color changes to a different categorical color, from green to yellow or orange. Visual determination thus becomes easy. A distance between metal layer 2 and metal layer 3, which is the height of determining part 8 in hollow area 4, is further preferably in a range not less than 400 nm and not greater than 1000 nm.

In the exemplary embodiment, electromagnetic source 11 is placed below metal layer 3, and incident electromagnetic wave 111 enters from the side of metal layer 3 of optical sensor 1, by using a light source typically electromagnetic source 11, and passes through to the side of metal layer 2. However, this may be reversed. In other words, metal layer 2 is disposed above electromagnetic source 11, and incident electromagnetic wave 111 enters from the side of metal layer 2 of optical sensor 1, using a light source typically electromagnetic source 11 and passes through to the side of metal layer 3. In other words, a structure is applicable to the exemplary embodiment as long as electromagnetic source 11 for supplying electromagnetic source and detector 12 for detecting a change of optical characteristic of electromagnetic wave supplied from electromagnetic source 11 are disposed facing each other with optical sensor 1 in between, and the electromagnetic wave supplied from electromagnetic source 11 passes through optical sensor 1 and is detected by detector 12.

### Second Exemplary Embodiment

Next, an optical sensor in the second exemplary embodiment is described with reference to FIG. 1.

Components of the optical sensor in the second exemplary embodiment are the same as that of optical sensor 1 described in the first exemplary embodiment, and thus description of components same as that in optical sensor 1 in the first exemplary embodiment is omitted.

The refractive index in hollow area 4 changes by aggregation of composite bodies 10 caused by the presence of analytes 80A in specimen 80. The optical sensor in the second exemplary embodiment is configured such that the center wavelength in the pseudo peak structure is practically across 570-nm to 590-nm bandwidth (yellow band) before and after the change of refractive index. An expression “practically across” is used for the descriptive purpose to indicate that the center wavelength at a peak of transmission spectrum before the refractive index changes is to the side of wavelength shorter than 570 nm (belonging to a green categorical color), and the center wavelength at a peak of transmission spectrum after change is to the side of wavelength longer (belonging to yellow or orange categorical color) than 580 nm (center of yellow band).

The center wavelength at the peak of transmission spectrum generated by optical interference of light reflected by gold configuring metal layer 2 and metal layer 3, in the state that capturing bodies 7 do not bind with analytes 80A, is called a first center wavelength.

The center wavelength at a peak of transmission spectrum generated by optical interference of light reflected by gold configuring metal layer 2 and metal layer 3, in the state that capturing bodies 7 and analytes 80A bind to form an aggregate of composite bodies 10, is called a second center wavelength.

Here, the next condition is satisfied: First center wavelength<570 nm<Second center wavelength.

More preferable relationship is to satisfy: First center wavelength<580 mm<Second center wavelength and at least First center wavelength<570 nm or 590 nm<Second center wavelength.

It is known that human eyes recognize a visible light color in successive change from purple, which is at the shortest wavelength end, to longer wavelength in order of blue, green, yellow, and red. When the presence of analyte 80A is detected based on a change of color defined by spectrum of reflected light, as in optical sensor 1 in the exemplary embodiment, it is important that to what extent the amount of perception of color change can be increased with respect to the same amount of change of wavelength.

When the human recognizes color, the human does not recognize simply according to an output ratio of three types of cone cells corresponding to red, green, and blue. Instead, it is known that colors in the same color system are recognized as a group. For example, diversifying red colors from red close to purple and red close to orange are recognized as a color category (categorical color) of red as a whole. This is called a categorical color perception. Accordingly, colors belonging to different categories are easy to distinguish in the successive color spectrum.

Color categories distinguished in categorical color perception have been studied from the aspect of linguistic culture (because colors that cannot be expressed by words cannot be used as categorical colors). As color names common to diversifying languages, red, yellow, green, blue, brown, pink, orange, white, gray, and black are defined as basic categorical color names.

For example, in case of monocolor light source with extremely narrow bandwidth, the categorical color changes from blue to green, yellow, orange, and red as the wavelength changes from the short to long wavelength side. However, a bandwidth of each color category is not equivalent. A change from blue to green is a gradual change in a band from approximately 400 nm to 570 nm. However, a change among three color categories of green, yellow, and orange is perceived just by crossing only a narrow 20-nm bandwidth from 570 nm to 590 nm (expressed as yellow).

Inventors focused on this relationship of categorical color perception and wavelength as a detection index of the optical sensor using visual detection of color change. In other words, if the center wavelength at a peak of transmission spectrum achieved for the first time by the structure shown in the first exemplary embodiment uses this change crossing the yellow band, the categorical color significantly changes (green and orange) by only a change of 20 nm. Therefore, visual detection of change becomes extremely easier than that in other wavelength bands.

The center wavelength at the peak of transmission spectrum can be set to this band by appropriately setting a distance between bottom face 2B of metal layer 2 and top face 3A of metal layer 3.

FIG. 7 shows results of inventors' study on changes of center wavelength at the peak of transmission spectrum by changing the height of hollow area 4 between metal layer 2 and metal layer 3 (distance between metal layer 2 and metal layer 3). A refractive index of deionized water is used as that in hollow area 4. It is apparent that the center wavelength at the peak of transmission spectrum linearly changes according to the height of hollow area 4.

In the exemplary embodiment, the center wavelength at the peak of transmission spectrum is set to 560 nm when deionized water (refractive index: 1.334) is filled in hollow area 4 by setting the height of determining part 8 to 820 nm based on results in FIG. 7. In this structure, the center wavelength at the peak of transmission spectrum shifted to 590 nm when the refractive index in determining part 8 is changed from deionized water to isooctane (refractive index: 1.39). As a result, the reflected color changes from green to orange, which is a different categorical color. This is an amount of change of effective refractive index that can be achieved by realizing 40% aggregation in a volume ratio to hollow area 4 when polystyrene latex beads are used for particles 9.

However, when the peak of center wavelength at the peak of transmission spectrum is set by a distance between metal layer 2 and metal layer 3, as described above, an optimum value changes depending on an amount of change of refractive index and its absolute value caused by aggregation of composite bodies 10 before and after reaction of capturing bodies 7 and analytes 80A. Since a bandwidth for yellow band is 20 nm, the amount of change of center wavelength, i.e., between the first center wavelength and the second center wavelength, is preferably at least 10 nm or more in order that the center wavelength at the peak of transmission spectrum practically crosses over the yellow band before and after reaction. If the amount of change of center wavelength is small, the center wavelength before reaction of capturing bodies 7 and analytes 80A belongs to a green categorical color and the wavelength needs to be long as much as possible. Accordingly, the height of determining part 8 in hollow area 4 needs to be further accurately controlled.

In addition, from the viewpoint of categorical color, a change from green to yellow is easier to detect than a change from yellow to orange. Accordingly, if the amount of change of center wavelength at the peak of transmission spectrum is not sufficiently large, the center wavelength before reaction is set to near the end of the longest wavelength (560 nm or shorter) and the center wavelength after reaction is preferably set to longer than 560 nm. In this case, although the center wavelengths at the peaks of transmission spectrums before and after reaction do not practically cross over the yellow band, a color change is detectable at high sensitivity even in the minimum change amount of center wavelength by a categorical color change from green to yellow.

The above discussion is described on the condition that the refractive index of particle 9 that determines the refractive index of determining part 8 is larger than the refractive index of solvent 80C, and thus the refractive index in determining part 8 always increases when aggregation of composite bodies 10 occur. However, the refractive index of particle 9 may be smaller than the refractive index of solvent 80C. In this case, the height of determining part 8 is set such that the center wavelength at the peak of transmission spectrum before reaction belongs to yellow or orange categorical color, and this changes to green categorical color after reaction.

### Third Exemplary Embodiment

Next is described an optical sensor in the third exemplary embodiment of the present invention with reference to FIG. 1. Components of the optical sensor in the exemplary embodiment are the same as that of optical sensor 1 described in the first exemplary embodiment. Therefore, description of components same as that of optical sensor 1 in the first exemplary embodiment is omitted.

In optical sensor 1 in the exemplary embodiment, electromagnetic source 11 is a light source of pseudo-monocolor or monocolor light with emission spectrum conforming to the center wavelength at the peak of the transmission spectrum caused by optical resonance of optical sensor 1, instead of white light source with broad wavelength band, such as sunlight in visible region and halogen lamp. For example, applicable light source includes monocolor LED light source (GaN system: Green, AlGaInP: Orange, etc.), organic EL light source, monocolor phosphor lamp using only monocolor phosphor (e.g., rare-earth phosphor with phosphor wavelength in green that is LaPO₄: Ce, Tb: abbreviated as LAP), and a consecutive spectrum light source such as halogen lamp to which a bandpass filter transmitting a predetermined wavelength band is added. A laser light source is also applicable. When the laser light source is used, the intensity of incident electromagnetic wave 111 can be extremely reinforced, and thus the thicknesses of metal layer 2 and metal layer 3 in determining part 8 can be further thickened. This can further reinforce optical interference, and thus the sensitivity can be improved by narrowing the peak width of transmission spectrum. In this case, to avoid a danger of injuring eyes, which is detector 12, depending on the intensity of transmitted electromagnetic wave 112, a frosted glass optical diffuser may be disposed between optical sensor 1 and detector 12.

An advantage of using electromagnetic source 11 with the above characteristic is described below.

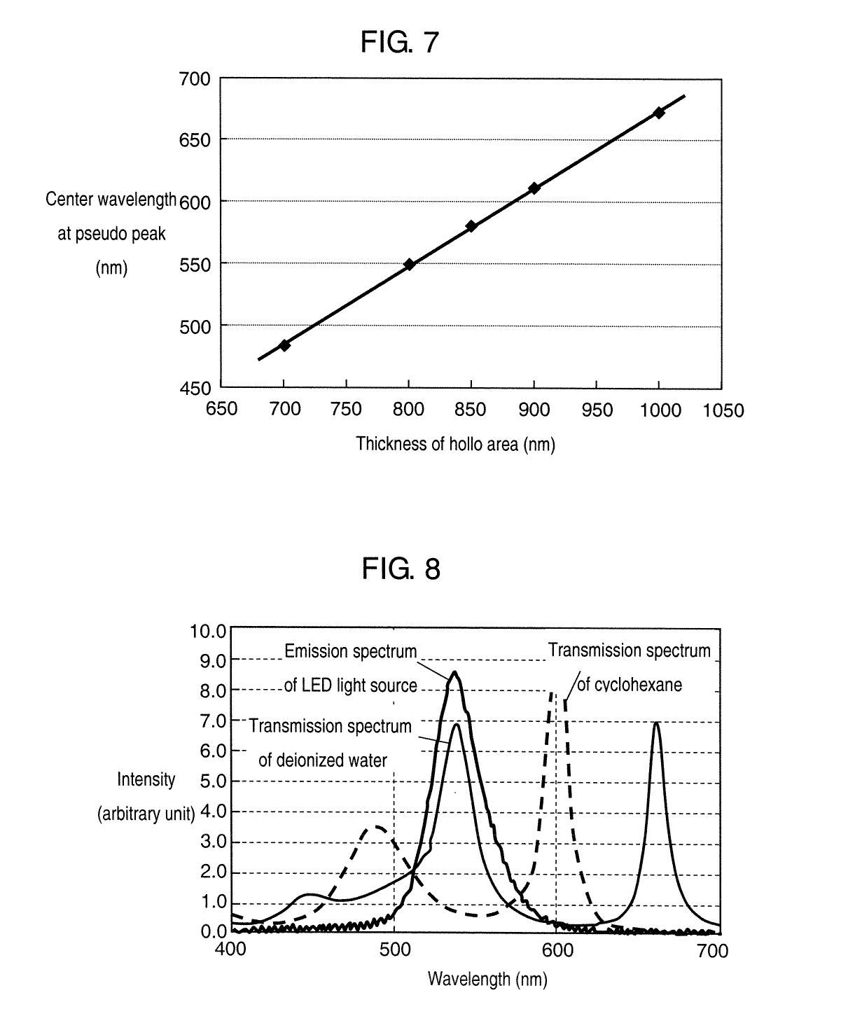

FIG. 8 is a graph (spectrums) created by overlaying emission spectrum of LED light source emitting pseudo-monocolor light with center wavelength of 536 nm (green) used as electromagnetic source 11 in this exemplary embodiment on the spectrum shown in FIG. 5 (transmission spectrum of optical sensor 1 when a halogen lamp is used as electromagnetic source 11). The solid line is the emission spectrum of an LED light source of electromagnetic source 11. The emission wavelength of LED light source is selected from transmission spectrums of optical sensor 1 in FIG. 8 in line with the peak wavelength of transmitted electromagnetic wave 112 when deionized water (effective refractive index: 1.33) is filled in hollow area 4.

When the effective refractive index in hollow area 4 is 1.33, which means specimen 80 does not contain analyte 80A and thus the effective refractive index in hollow area 4 does not increase due to aggregation of composite bodies 10, the center wavelength at the peak of transmission spectrum of transmitted electromagnetic wave 112 transmitted in optical sensor 1 conforms to the emission spectrum of LED light source, which is electromagnetic source 11. In other words, incident electromagnetic wave 111 from electromagnetic source 11 mostly passes through optical sensor 1 and is detected by detector 12.

However, when optical sensor 1 has transmission spectrum shown by the dotted line in FIG. 8, which means specimen 80 contains analyte 80A and the effective refractive index of hollow area 4 is increased due to aggregation of composite bodies 10, the peak wavelength of transmission spectrum of optical sensor 1 does not include an emission spectrum component of the LED light source, which is electromagnetic source 11. Therefore, incident electromagnetic wave 111 from electromagnetic source cannot pass through optical sensor 1, and thus transmitted electromagnetic wave 112 is not detected or detected with reduced intensity by detector 12.

This means that a green light of transmitted electromagnetic wave 112 from optical sensor 1 that has been noticed becomes invisible when specimen 80 contains analyte 80A, in case of visual observation by eyes at detector 12. In other words, a detection index for determination becomes the presence of light or gradation of light passing through optical sensor 1 from electromagnetic source 11, instead of the detection index depending on a color change used in the first and second exemplary embodiments.

The sensitivity to color is not always same for all individuals and races. In addition, there are people with impaired color vision who have inherited or acquired weakness to color determination. In particular, a shift from around 550 nm (green) to 600 nm (orange) mainly used in the present invention is a color range particularly difficult for people with impaired color vision, which is at a relatively high rate, to distinguish.

On the other hand, the exemplary embodiment enables to determine based on light and dark of light transmitted through optical sensor 1, instead of a color change. This eliminates an influence of personal difference in color identification, and thus allows to provide a highly versatile inspection method.

In the exemplary embodiment, detector 12 is visual inspection by eyes. However, detector 12 may be an optical detector that detects the intensity of transmitted electromagnetic wave 112, such as a photo diode. In the structures of the first exemplary embodiment and second exemplary embodiment, a color change in line with a spectral change of transmitted electromagnetic wave 112 or spectral change itself needs to be directly observed. Therefore, optical sensor 1 needs to be equipped with a spectrometer when a physical detector other than eyes is used. However, in the exemplary embodiment, what is observed is emission spectrum of electromagnetic source 11 or the light intensity of wavelength equivalent to transmission spectrum peak of optical sensor 1. Therefore, no spectroscopic function is needed in detector 12. However, also in this case, it would be better if optical sensor 1 is equipped with a filter for removing light with wavelengths other than that equivalent to emission spectrum of electromagnetic source 11 and the transmission spectrum peak of optical sensor 1, in order to avoid influence of external light.

The exemplary embodiment uses an LED light source that emits monocolor light with wavelength of 536 nm as electromagnetic source 11. However, the exemplary embodiment is not limited to this wavelength. Applicable light sources include a monocolor or pseudo-monocolor light source with wavelengths approximately conforming to the transmission spectrum peak of optical sensor 1 before the refractive index in hollow area 4 changes due to aggregation of composite bodies 10 and practically excluding the transmission spectrum peak of optical sensor 1 after the effective refractive index in hollow area 4 changes due to aggregation of composite bodies 10. The center wavelength at the peak of transmission spectrum of optical sensor 1 is determined by the effective refractive index in hollow area 4 and the height of determining part 8 of hollow area 4. Therefore, if the height of determining part 8 of hollow area 4 differs, an equivalent effect is achievable by using electromagnetic source 11 with wavelength suited to that height.

Still more, wavelength of electromagnetic wave irradiated from electromagnetic source 11 is preferably variable. This enables to maintain the detection sensitivity by fine adjustment of wavelength of incident electromagnetic wave 111 to absorb variations in the center wavelength at the peak of transmission spectrum due to manufacturing variations in the height of hollow area 4 of optical sensor 1.

### Fourth Exemplary Embodiment

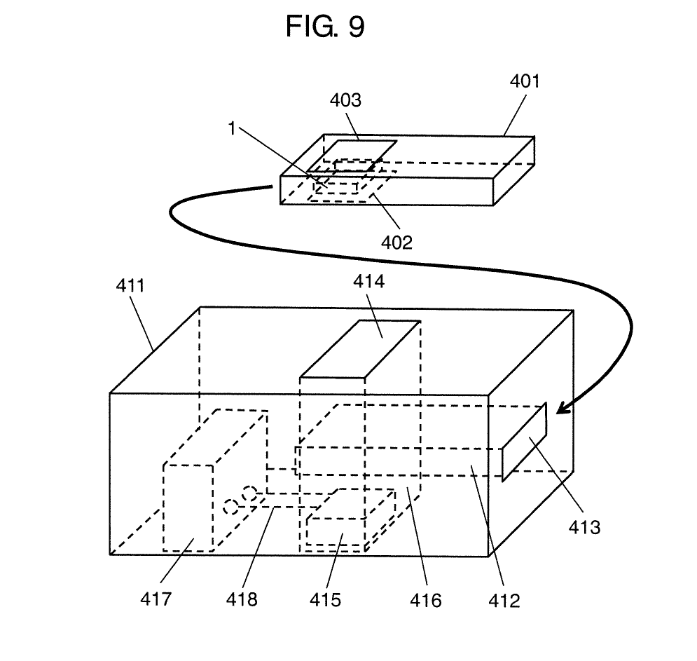

Next, the fourth exemplary embodiment of the present invention is described with reference to FIG. 9.

The exemplary embodiment is inspection body 401 and inspection unit 411 that intend to increase the detection accuracy by optimally configuring an optical path connecting electromagnetic source 11, optical sensor 1, and detector 12 in addition to housing optical sensor 1 and using an appropriate light source as electromagnetic source 11, as described above.

FIG. 9 is a schematic view of a structure of inspection body 401 and inspection unit 411 in the exemplary embodiment.

Inspection body 401 is a resin cartridge for housing optical sensor 1 inside, and includes light inlet 402 and light outlet 403. Light entering from light inlet 402 passes through optical sensor 1, and is discharged from light outlet 403 to outside inspection body 401. Inspection body 401 may have a passage structure (not illustrated) for introducing a specimen solution from outside to optical sensor 1.

Inspection unit 411 includes light source unit 415 inside, which is electromagnetic source 11. Light source unit 415 is a green LED light source of IGaN/GaN system with center wavelength of 550 nm. Light source unit 415 illuminates by receiving power from power unit 417 via power supply line 418. Power unit 417 is a primary battery, such as dry batter; a secondary battery, such as nickel hydride battery; or commercial power supply. Light from light source unit 415 is irradiated outside from observation opening 414 through optical path 416, and reaches detector 12 (not illustrated) at an upper part.

Inspection unit 411 includes hollow inspection body case 412 communicated with outside by opening 413. Inspection body 401 is inserted to inspection body case 412 from opening 413. Inspection body case 412 is disposed such that it crosses optical path 416. In the state inspection body 401 is housed in inspection body case 412, light irradiated from light source unit 415 passes in optical path 416, enters inspection body case 412, and is irradiated to optical sensor 1 from light inlet 402 of inspection body 401. Light transmitted in optical sensor 1 passes through light outlet 403, and is discharged outside from observation opening 414.

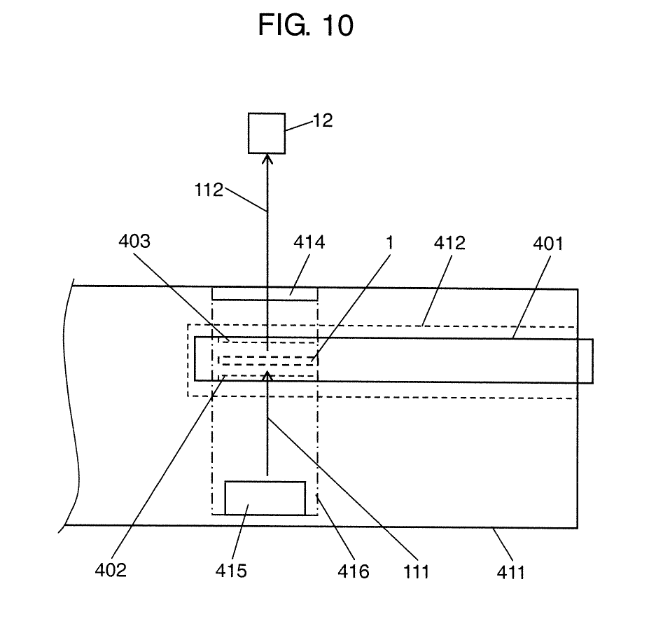

FIG. 10 schematically illustrates a path of incident electromagnetic wave 111, which is a light irradiated from light source unit 415, until it reaches detector 12 after passing through inspection body 401. Incident electromagnetic wave 111 irradiated from light source unit 415 into optical path 416 enters optical sensor 1 inside inspection body 401 from light inlet 402. Optical sensor 1 operates in the same way as that described in the third exemplary embodiment. Transmitted wavelength of optical sensor 1 and the center wavelength of emission spectrum of light source unit 415 are designed to approximately conform in the state the refractive index in hollow area 4 does not change in optical sensor 1. Accordingly, incident electromagnetic wave 111 irradiated from light source unit 415 becomes transmitted electromagnetic wave 112 after passing through optical sensor 1, and is irradiated inside optical path 416 again from light outlet 403 of inspection body 401. Transmitted electromagnetic wave 112 is irradiated outside from observation opening 414 of inspection unit 411, and reaches detector 12. If the transmitted wavelength of optical sensor 1 is shifted due to a change of effective refractive index in hollow area 4, the emission spectrum of incident electromagnetic wave 111 irradiated from light source unit 415 and transmitted wavelength of optical sensor 1 do not match. Accordingly, the intensity of transmitted electromagnetic wave 112 observed at detector 12 reduces, or preferably becomes almost none.

As shown in FIG. 10, a straight line connecting light source unit 415 and detector 12 is designed to perpendicularly cross optical sensor 1. In other words, incident electromagnetic wave 111 is at right angle to optical sensor 1. This means incident electromagnetic wave 111 enters such that θ in Formula (1) and Formula (2) becomes zero. By affixing the positional and angular relationship of light source unit 416, optical sensor 1, and detection 12; a change of interference condition in optical sensor 1 that may be caused by a change of incident angle of incident electromagnetic wave 111 and observation angle of transmitted electromagnetic wave 112 can be eliminated. This improves reliability of inspection.

In the exemplary embodiment, a straight line connecting light source unit 415 and detector 12 perpendicularly crosses optical sensor 1. However, the exemplary embodiment is not always limited to this structure. By appropriately designing θ in Formulae (1) and (2), the same effect is achievable at other angles.

The state that a straight line connecting light source unit 415 and detector 12 perpendicularly crosses optical sensor 1 means the state that any plane including the straight line connecting light source unit 415 and detector 12 perpendicularly crosses at least metal layer 2 or metal layer 3 configuring optical sensor 1.

Still more, in the exemplary embodiment, a light source of light source unit 415 is an LED light source. However, since it is not perfectly coherent, although the light emitted from the LED light source is characterized by its relatively high directionality, components that enter optical sensor 1 at undesired angles always exist even if the above positions are achieved. In addition, since observation opening 414 has a limited size, it is not easy to make the straight line connecting light source unit 415 and detector 12 perpendicularly cross optical sensor 1 accurately, particularly when detector 12 is visual inspection by eyes. These components at undesired angles do not satisfy the designed interference condition in optical sensor 1, and thus give detrimental effect on observation at detector 12. More specifically, an amount of change of the intensity of transmitted electromagnetic wave 112 reduces.

To avoid such influence, a smaller area of observation opening 414 and a smaller cross section in a face parallel to observation opening 414 of optical path 416 are preferable. However, this smaller area leads to a smaller area of optical sensor 1 that can be used for inspection. Accordingly, optimum design is needed.

The inner surface of optical path 416 is preferably treated to reduce reflectivity to the wavelength of emission spectrum of light source unit 415. This is to suppress entry of reflection of light irradiated from light source unit 415 at undesired angles to optical sensor 1. Such treatment is, for example, painting by mat black paint and attachment of flocked processed paper.

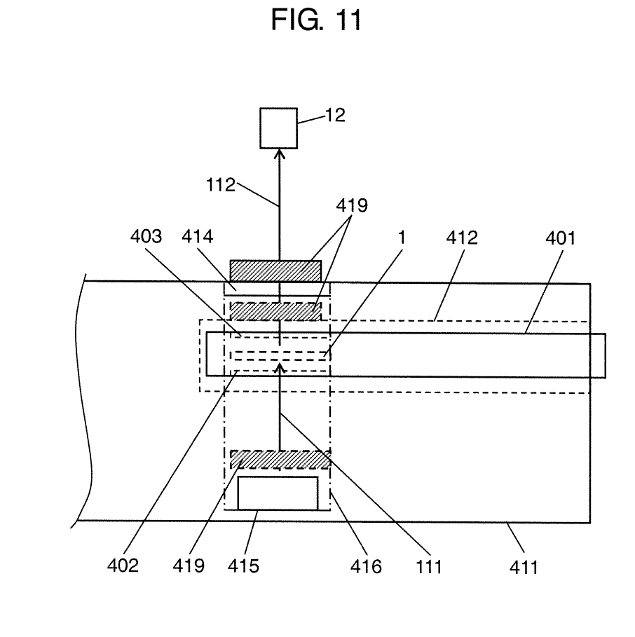

As shown in FIG. 11, collimator 419 for aligning a light irradiated from light source unit 415 close to a parallel light having a directional vector perpendicular to optical sensor 1 may be provided at least between light source unit 415 and detector 401, between detector 401 and observation opening 414, or between observation opening 414 and detector 12.

Specific examples of collimator 419 are a collimator lens formed by combining convex lenses (Kepler system) or convex and concave lenses (Galileo system), and a louver formed of multiple thin sheets parallel to the perpendicular direction of optical sensor 1. The use of collimator lens enables to create a parallel light without losing its light quantity even if a distance from light source unit 415 or observation opening 414 is short. On the other hand, the use of louver is preferable with respect to relatively inexpensive cost because it is a mechanical structure that does not need an optical component. In addition, a reflective light system, such as a parabolic mirror, may be assembled in light source unit 415. Or, an optical waveguide may be used as the collimator. The optical waveguide is, for example, to link light source unit 415, inspection body 401 of optical sensor 1 housed in inspection body case 412, and observation opening 414 as an optical waveguide, using an optical element, such as a light pipe and imaging fiber (a bundle of optical fiber for connecting the inlet and outlet in one-to-one relation). The optical waveguide is preferable because it has a high collimating effect and small loss of light quantity. It is also preferable because it is not affected by stray light and thus color contrast can be increased.

By the use of such collimator 419, transmitted electromagnetic wave 112 can be observed only when detector 12 is disposed at a preferable angle relative to light source unit 415 and optical sensor 1. The inspection accuracy can thus be improved.

Still more, the exemplary embodiment can always use light with appropriate spectrum. Accordingly, inspection can always take place under optimum conditions without being restricted by the place of use of inspection unit 411.

Furthermore, the use of a light source with variable wavelength as light source unit 415 enables to adjust the emission wavelength of light source unit 415 against variations in the interference condition typically caused by variations in the height of hollow area 4 in optical sensor 1. This enables to always match the wavelength of incident electromagnetic wave 111 to the transmitted wavelength of optical sensor 1. Accordingly, restrictions related to assembly accuracy of optical sensor 1 can be eased to improve the yield and reduce costs.

In the first, second, third, and fourth exemplary embodiments, terms indicating directions, such as top face, bottom face, above, and below, are relative directions dependent only on relative positional relationship of components of the optical sensor, and thus they are not absolute directions, such as a vertical direction.

### INDUSTRIAL APPLICABILITY

The present invention offers a small optical sensor with simple structure. Accordingly, it is applicable to small and low-cost biosensors and chemical sensors.

### REFERENCE MARKS IN THE DRAWINGS

1 Optical sensor
2 Metal layer (first metal layer)
3 Metal layer (second metal layer)
4 Hollow area
5 Attaching part (first attaching part)
6 Attaching part (second attaching part)
7 Capturing body
8 Determining part
9 Particle
10 Composite body
11 Electromagnetic source (light source)
12 Detector
13 Affixing part
80 Specimen
80A Analyte (target substance to be detected)
80B Other solute
80C Solvent
111 Incident electromagnetic wave
112 Transmitted electromagnetic wave
401 Inspection body
402 Light inlet
403 Light outlet
411 Inspection unit
412 Inspection body case
413 Opening
414 Observation opening
415 Light source unit
416 Optical path
417 Power unit
418 Power supply line

1. An optical sensor comprising:
a first metal layer having a first top face and a first bottom face;
a second metal layer having a second top face and a second bottom face; and
a hollow area sandwiched by the first metal layer and the second metal layer, the hollow area being an area where a capturing body for capturing a target substance to be detected can be placed,
wherein
the first bottom face of the first metal layer faces the second top face of the second metal layer,
a thickness of the first metal layer and a thickness of the second metal layer are not less than 5 nm and not greater than 50 nm,
the hollow area includes a determining part for determining a presence of the target substance contained in a specimen,
the second metal layer can transmit an electromagnetic wave from the second bottom face to the second top face, and
the first metal layer can transmit an electromagnetic wave from the first bottom face to the first top face.

2. The optical sensor of claim 1,
wherein
the hollow area further includes a affixing part where the capturing body is placed.

3. The optical sensor of claim 1,
wherein
the capturing body is physically adsorbed on at least one of the first bottom face of the first metal layer and the second top face of the second metal layer in the determining part.

4. The optical sensor of claim 1,
wherein
a distance between the first metal layer and the second metal layer is not less than 400 nm and not greater than 1600 nm.

5. The optical sensor of claim 1,
wherein
a distance between the first metal layer and the second metal layer is not less than 400 nm and not greater than 1000 nm.

6. The optical sensor of claim 1,
wherein
thicknesses of the first metal layer and the second layer are not less than 5 nm and not greater than 30 nm.

7. The optical sensor of claim 1,
wherein
the first metal layer and the second metal layer are both formed of gold.

8. The optical sensor of claim 1,
wherein
in a center wavelength at a peak of transmission spectrum that appears due to an optical interference caused by a light passing through the first metal layer and the second metal layer,
a first center wavelength in a state that the capturing body does not capture the target substance and a second center wavelength in a state that the capturing body captures the target substance establish a relationship: First center wavelength<580 nm<Second center wavelength, and
at least one of conditions of (1) First center wavelength<570 nm and (2) Second center wavelength>590 nm is satisfied.

9. The optical sensor of claim 1,
wherein
in a center wavelength at a peak of transmission spectrum that appears due to an optical interference caused by a light passing through the first metal layer and the second metal layer,
a first center wavelength in a state that the capturing body does not capture the target substance satisfies a condition of First center wavelength<570 nm, and
a second center wavelength in a state that the capturing body captures the target substance satisfies a condition of Second center wavelength>570 nm.

10. The optical sensor of claim 1,
wherein
the capturing body is affixed on a surface of a particle.

11. A detection method comprising:
preparing an optical sensor including:
a first metal layer having a first top face and a first bottom face;
a second metal layer having a second top face and a second bottom face; and
a hollow area sandwiched by the first metal layer and the second metal layer, the hollow area being an area where a capturing body for capturing a target substance to be detected can be placed,
wherein
the first bottom face of the first metal layer faces the second top face of the second metal layer,
a thickness of the first metal layer and a thickness of the second metal layer are not less than 5 nm and not greater than 50 nm, and
the hollow area includes a determining part for determining a presence of the target substance contained in a specimen;
inserting the specimen into the hollow area by using a capillary action;
entering an electromagnetic wave from the second bottom face of the second metal layer; and
detecting the electromagnetic wave passing through the first metal layer.

12. A method for affixing a capturing body, the method comprising:
a first step of inserting a solute, containing a capturing body for capturing a target substance to be detected, in a hollow area of an optical sensor, the optical sensor including:
a first metal layer having a first top face and a first bottom face;
second metal layer having a second top face and a second bottom face; and
the hollow area sandwiched by the first metal layer and the second metal layer, the hollow area being an area where the capturing body for capturing the target substance can be placed,
wherein
the first bottom face of the first metal layer faces the second top face of the second metal layer,
a thickness of the first metal layer and a thickness of the second metal layer are not less than 5 nm and not greater than 50 nm, and
the hollow area includes a determining part for determining a presence of the target substance contained in a specimen; and
a second step of drying the solute and placing the capturing body in the hollow area after the first step.

13. The method for affixing a capturing body of claim 12, wherein
the capturing body is fixed on a surface of a particle.

14. An inspection unit into which the optical sensor of claim 1 is inserted for use, the inspection unit comprising:
an opening;
an optical sensor case for receiving the optical sensor inserted from the opening;
an electromagnetic source for irradiating an electromagnetic wave to the optical sensor; and
an optical path for guiding a light irradiated from the electromagnetic source through the optical sensor at a predetermined angle to outside the inspection unit.

15. The inspection unit of claim 14,
wherein
the electromagnetic source has one of monocolor or pseudo-monocolor emission spectrum including an interference wavelength relative to a transmitted light of the optical sensor that is determined by a distance between the first metal layer and the second metal layer and a refractive index in the hollow area of the optical sensor.

16. The inspection unit of claim 14 further comprising:
a collimator disposed at any position in the optical path for restricting an angle of the light irradiated from the electromagnetic source approximately to a given angle relative to the optical sensor.

17. The inspection unit of claim 16,
wherein
the collimator is a louver having multiple planes parallel to a straight line linking the electromagnetic source and the optical sensor at the predetermined angle.

18. The inspection unit of claim 16,
wherein
the collimator is a combination of a plurality of convex lenses and concave lenses.

19. The inspection unit of claim 16,
wherein
the collimator is an optical waveguide covering the optical path.
